(19) United States
(12) Patent Application Publication
(10) Pub. No.: US 2019/0198149 A1
(43) Pub. Date: Jun. 27, 2019

(54) DETERMINING ACTIONABLE EVENTS FOR GAMIFIED HEALTH GOALS BASED UPON USER COMMUNICATIONS

(71) Applicant: International Business Machines Corporation, Armonk, NY (US)

(72) Inventors: Paul R. Bastide, Boxford, MA (US); Matthew E. Broomhall, Goffstown, NH (US); Liam S. Harpur, Skerries (IE); Lin Sun, Morrisville, NC (US)

(21) Appl. No.: 15/851,982

(22) Filed: Dec. 22, 2017

(51) Int. Cl. G16H 20/30

(52) U.S. Cl. CPC G16H 20/30

(57) ABSTRACT

Attaining health goals based upon user communications can include extracting, using computer hardware, a plurality of conversation elements from communications of a user and matching, using the computer hardware, the plurality of conversation elements to activities of the user that are correlated with a gamified health goal of the user. Using the computer hardware, one or more of the activities matched to selected ones of the plurality of conversation elements having an affinity to the gamified health goal that exceeds an affinity threshold can be determined. The one or more activities can be provided, using the computer hardware, as an elaboration of the gamified health goal.

### RESERVATION OF RIGHTS IN COPYRIGHTED MATERIAL

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

This disclosure relates to achieving health goals based upon user communications. The term “gamification” refers to the use of game mechanics and user experience design from computer-based games to digitally engage and motivate people to achieve their goals. These goals may vary widely. In some cases, the goal is fitness related. In other cases, the goal is to change a user behavior. In still other cases, the goal is to develop a skill. Examples of “gamification” of a goal are where an application attempts to motivate a user to achieve the goal through the use of the accumulation of points, badges, streaks, and/or leaderboards. The use of gamification allows a device or a system to digitally engage with the user and provide motivation rather than the user interacting with another user.

### SUMMARY

In one or more embodiments, a computer-implemented method can include extracting, using computer hardware, a plurality of conversation elements from communications of a user and matching, using the computer hardware, the plurality of conversation elements to activities of the user that are correlated with a gamified health goal of the user. The method can include determining, using the computer hardware, at least one of the activities matched to selected ones of the plurality of conversation elements having an affinity to the gamified health goal that exceeds an affinity threshold. The method can also include providing, using the computer hardware, the at least one of the activities as an elaboration of the gamified health goal.

In one or more embodiments, a system includes a memory configured to store program code and a processor coupled to the memory. The processor, in response to executing the program code, is configured to initiate operations including extracting a plurality of conversation elements from communications of a user and matching the plurality of conversation elements to activities of the user that are correlated with a gamified health goal of the user. The operations can include determining at least one of the activities matched to selected ones of the plurality of conversation elements having an affinity to the gamified health goal that exceeds an affinity threshold. The operations can also include providing the at least one of the activities as an elaboration of the gamified health goal.

In one or more embodiments, a computer program product includes a computer readable storage medium having program instructions stored thereon. The program instructions are executable by computer hardware to initiate operations. The operations can include extracting a plurality of conversation elements from communications of a user and matching the plurality of conversation elements to activities of the user that are correlated with a gamified health goal of the user. The operations can include determining at least one of the activities matched to selected ones of the plurality of conversation elements having an affinity to the gamified health goal that exceeds an affinity threshold. The operations can also include providing the at least one of the activities as an elaboration of the gamified health goal.

This Summary section is provided merely to introduce certain concepts and not to identify any key or essential features of the claimed subject matter. Other features of the inventive arrangements will be apparent from the accompanying drawings and from the following detailed description.

### BRIEF DESCRIPTION OF THE DRAWINGS

The inventive arrangements are illustrated by way of example in the accompanying drawings. The drawings, however, should not be construed to be limiting of the inventive arrangements to only the particular implementations shown. Various aspects and advantages will become apparent upon review of the following detailed description and upon reference to the drawings.

FIG. 1 depicts an example of a network computing system for use with one or more embodiments described within this disclosure.

FIG. 2 depicts an example architecture for computer hardware for use with one or more embodiments described within this disclosure.

FIG. 3 depicts an example method for elaborating on a gamified health goal based upon user communications.

FIG. 4 depicts an example of a user interface for use with one or more embodiments described within this disclosure.

### DETAILED DESCRIPTION

While the disclosure concludes with claims defining novel features, it is believed that the various features described within this disclosure will be better understood from a consideration of the description in conjunction with the drawings. The process(es), machine(s), manufacture(s) and any variations thereof described herein are provided for purposes of illustration. Specific structural and functional details described within this disclosure are not to be interpreted as limiting, but merely as a basis for the claims and as a representative basis for teaching one skilled in the art to variously employ the features described in virtually any appropriately detailed structure. Further, the terms and phrases used within this disclosure are not intended to be limiting, but rather to provide an understandable description of the features described.

This disclosure relates to achieving health goals based upon user communications and, more particularly, to elaborating a gamified health goal by determining activities for achieving the gamified health goal based upon user communications. In accordance with the inventive arrangements described herein, a system is capable of helping a user reach one or more established health goals through an analysis of the user's communications. The health goals may be gamified health goals. The system is capable of analyzing the user's communications over time and extracting conversation elements, e.g., “tokens”, from the communications. The system is capable of matching the conversation elements with various activities of the user. The activities may be activities that the user has performed historically. The activities are associated with various health goals and gamified health goals of the user.

The system is capable of determining actionable events that the user may perform in order to reach a particular gamified health goal not yet achieved by the user. For example, for a given gamified health goal, the system is capable of suggesting one or more activities that, if performed by the user, would either allow the user to reach the gamified health goal or move the user closer to achieving the gamified health goal. Each of the activities may be presented as an actionable event that the system has matched with the gamified health goal of the based upon the extracted conversation elements. By matching the conversation elements of the user's communications with activities typically performed by the user, the system is capable of providing suggestions for reaching the user's gamified health goal by engaging in selected activities in which the user typically, or historically, engages.

Further aspects of the embodiments described within this disclosure are described in greater detail with reference to the figures below. For purposes of simplicity and clarity of illustration, elements shown in the figures have not necessarily been drawn to scale. For example, the dimensions of some of the elements may be exaggerated relative to other elements for clarity. Further, where considered appropriate, reference numbers are repeated among the figures to indicate corresponding, analogous, or like features.

FIG. 1 depicts an example of a network computing system 100 for use with one or more embodiments described within this disclosure. Network computing system 100 contains devices 110, 115, and 120, an optional messaging system 125, an optional social networking system 130, and a goal achievement system 135. The noted elements are interconnected, e.g., communicatively linked, by a network 105.

Network 105 is the medium used to provide communication links between various devices and data processing systems connected together within network computing system 100. Network 105 may include connections, such as wired communication links, wireless communication links, or fiber optic cables. Network 105 may be implemented as, or include, one or more or any combination of different communication technologies such as a Wide Area Network (WAN), a Local Area Network (LAN), a wireless network (e.g., a wireless WAN and/or a wireless LAN), a mobile or cellular network, a Virtual Private Network (VPN), the Internet, the Public Switched Telephone Network (PSTN), and so forth.

Devices 110, 115, and 120 are capable of coupling to network 105 via wired and/or wireless communication links. Devices 110 and/or 120 may be implemented as, for example, personal computers, portable computing or communication devices, network computers, tablet computers, and/or mobile phones. Device 110 may be implemented as one type of device, while device 120 is implemented as another different type of device. Device 115 may be implemented as a wearable device. For example, device 115 may be implemented as a smart watch, a wearable monitor (e.g., a health monitor capable of measuring one or more vital signs such as heart rate, blood pressure, etc.), or a fitness device such as a step tracker. Device 115 is capable of communicating with device 110 via a wired or wireless communication link.

As defined herein, the term “communication,” means any of a variety of messages that are capable of being sent and/or received through network 105. Examples of communications include, but are not limited to, electronic mail (email), instant messages, short message service (SMS) messages, text messages, posts to a Website and/or social networking system 130, and/or messages posted to chat sessions.

Messaging system 125, which is optional, is capable of coupling to network 105 via wired and/or wireless communication links. In one aspect, messaging system 125 is implemented as one or more interconnected computer systems, e.g., servers. Messaging system 125 is capable of executing suitable operational software to support the conveyance of messages (e.g., text, SMS, instant, and/or email) among coupled devices such as devices 110, 115, and/or 120.

Social networking system 130, which is optional, is capable of coupling to network 105 through wired and/or wireless communication links. Social networking system 130 is implemented as one or more interconnected computer systems, e.g., servers. As defined herein, a “social networking system” is a computing platform that allows users to build social networks or social relations among people who share similar interests, activities, backgrounds or real-life connections. Through a social networking system, users may send communications through different mechanisms such as by posting messages or other media, commenting on messages, posts, or other media, replying to messages, and performing other operations such as “liking” a communication or item of media, sharing the communication or item of media, expressing an emotional sentiment, and so forth. In the context of social networking system 130, actions such as posting, replying, liking, sharing, expressing sentiment, and so forth are programmatic actions that are monitored and persisted therein, e.g., within a data storage device in a data structure within and/or accessible by, social networking system 130.

As an illustrative and nonlimiting example, user A may use device 110 and/or device 115 to communicate with user B using device 120. Communications exchanged between user A and user B (e.g., between devices 115 and 120 or devices 110 and 120) may be any of a variety of different messages as described herein that may be conveyed through network 105 using messaging system 125 and/or social networking system 130. Goal achievement system 135 is capable of analyzing communications of user A, which may include messages sent from user A and/or messages received by user A. Thus, the communications analyzed by goal achievement system 135 may include messages exchanged with between user A and user B and messages exchanged between user A and one or more other users.

In one or more embodiments, goal achievement system 135 retrieves communications of user A from messaging system 125 and/or social networking system 130. In particular embodiments, goal achievement system 135 is capable of performing Natural Language Processing (NLP) on retrieved communications for user A. NLP is a field of computer science, artificial intelligence, and linguistics which implements computer processes to facilitate interactions between computer systems and human (natural) languages. NLP enables computers to derive computer-understandable meaning from natural language input. The International Organization for Standardization (ISO) publishes standards for NLP, one such standard being ISO/TC37/SC4. As part of NLP, semantic analysis may be performed. Semantic analysis may follow parsing and involve extraction of context-independent aspects of a sentence's meaning, including entities, named entities, the semantic roles of entities mentioned in the sentence, and quantification information, such as cardinality, iteration, and dependency.

Using the processing techniques described, goal achievement system 135 is capable of extracting one or more tokens from the communications of user A. Goal achievement system 135 is capable of matching the tokens, also referred to herein as “conversation elements”, with activities of user A. These activities may be activities historically performed by user A. These activities may also be correlated with various health goals and/or gamified health goals of user A.

Goal achievement system 135 is capable of suggesting one or more actionable events that may be performed by user A to achieve, or move closer to, a gamified health goal of user A that has not yet been achieved. The actionable events may represent different ways to achieve a particular gamified health goal. For example, the actionable events specify selected ones of the activities. Goal achievement system 135 is capable of determining the actionable events and providing the gamified health goal to user A in a format where the gamified health goal is broken out into, or elaborated, as one or more actionable events (e.g., via device 110 and/or device 115).

FIG. 1 is provided for purposes of illustration and is not intended to limit the inventive arrangements described herein. It should be appreciated that network computing system 100 may include fewer elements than shown or more elements than shown. For example, network computing system 100 may include fewer or more servers, clients, and/or other devices. In addition, one or more of the elements illustrated in network computing system 100 may be merged or combined.

In addition, while the example embodiments described within this disclosure are illustrated using a centralized system, e.g., goal achievement system 135, in one or more other embodiments, the operations attributed to goal achievement system 135 may be performed by a device such as device 110 or device 115.

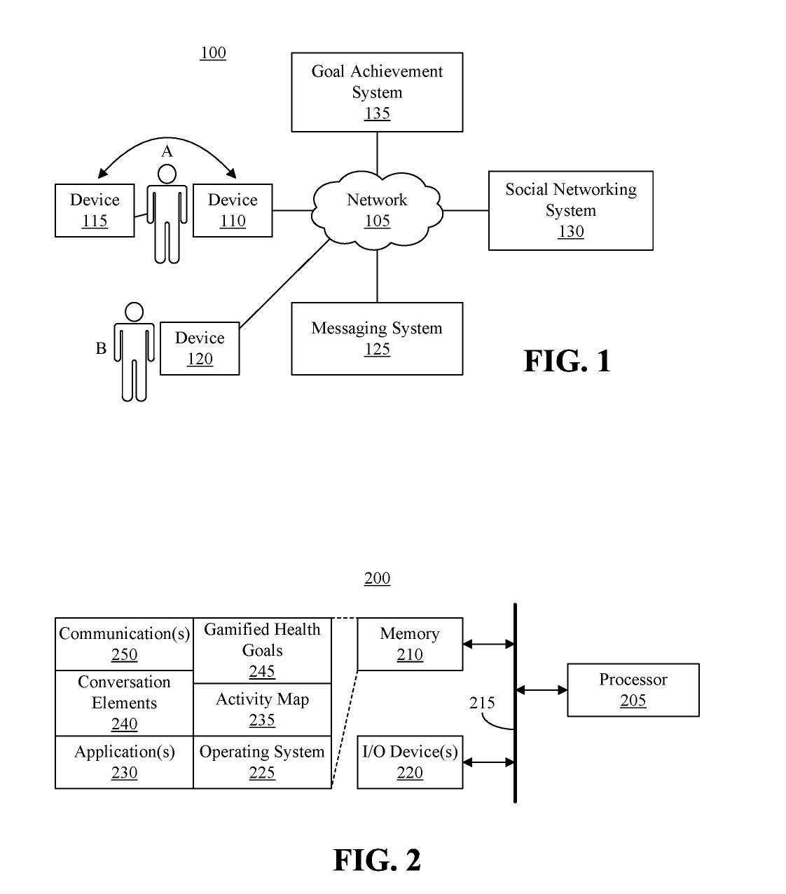

FIG. 2 depicts an example architecture 200 for computer hardware for use with one or more embodiments described within this disclosure. Architecture 200 may be used to implement a computer or other system or device that is suitable for storing and/or executing program code. In one or more embodiments, architecture 200 may be used to implement goal achievement system 135. In one or more other embodiments, architecture 200 may be used to implement device 110 and/or device 115.

Architecture 200 includes at least one processor 205. Processor 205 is coupled to memory 210 through interface circuitry 215. Architecture 200 stores computer readable instructions (also referred to as “program code”) within memory 210. Memory 210 is an example of computer readable storage media. Processor 205 executes the program code accessed from memory 210 via interface circuitry 215.

Memory 210 includes one or more physical memory devices such as, for example, a local memory and one or more bulk storage devices. The term “local memory” refers to non-persistent memory device(s) generally used during actual execution of the program code. Examples of local memory include random access memory (RAM) and/or any of the various types of RAM that are suitable for use by a processor for executing program code. The term “bulk storage device” refers to persistent data storage devices. Examples of a bulk storage device include a hard disk drive (HDD) and a solid-state drive (SSD). Architecture 200 may also include one or more cache memories (not shown) that provide temporary storage of at least some program code to reduce the number of times program code must be retrieved from a bulk storage device during execution.

Examples of interface circuitry 215 include, but are not limited to, an input/output (I/O) subsystem, an I/O interface, a bus system, and a memory interface. For example, interface circuitry 215 may be one or more of any of several types of bus structures, including a memory bus or memory controller, a peripheral bus, an accelerated graphics port, and a processor or local bus using any of a variety of bus architectures. By way of example, and not limitation, such bus architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, and PCI express (PCIe).

Architecture 200 further may include one or more input/output (I/O) devices 220 coupled to interface circuitry 215. I/O devices 220 may be coupled to architecture 200, e.g., interface circuitry 215, either directly or through intervening I/O controllers. Examples of I/O devices 220 include, but are not limited to, a keyboard, a display device, a pointing device, one or more communication ports, a network adapter, etc. A network adapter refers to circuitry that enables architecture 200 to become coupled to other systems, computer systems, remote printers, and/or remote storage devices through intervening private or public networks. Modems, cable modems, Ethernet cards, and wireless transceivers are examples of different types of network adapters that may be used with architecture 200.

Examples of wireless transceivers can include, but are not limited to, radio frequency transceivers and optical (e.g., infrared) transceivers. The specific design and implementation of the wireless transceivers can depend on the particular type of device and/or system implemented using architecture 200 and/or the type of communication network(s) over which such a device and/or system is intended to operate.

As illustrative and non-limiting examples, a wireless transceiver may be designed to operate over one or more mobile networks (e.g., GSM, GPRS, EDGE), a WiFi network which may include a WiMax network, a short-range wireless network (e.g., a Bluetooth network), and/or any combination of the foregoing.

Memory 210 stores one or more program modules. The program modules may generally include routines, programs, objects, components, logic, data structures, and so on. In one or more embodiments, memory 210 stores an operating system 225 and one or more application(s) 230. Memory 210 may also store data. In an aspect, operating system 225 and application(s) 230, being implemented in the form of executable program code, are executed by architecture 200 and, more particularly, by processor 205, to perform the operations described within this disclosure. As such, operating system 225 and application(s) 230 may be considered an integrated part of architecture 200. It should be appreciated that any data used, generated, and/or operated upon by a system implemented using architecture 200 (e.g., processor 205) are functional data structures that impart functionality when employed as part of the system.

In the example of FIG. 2, memory 210 is capable of storing an activity map 235, conversation elements 240 (e.g., tokens), one or more gamified health goals 245, and communications 250. Communications 250 may be retrieved from one or more other sources such as one or more messaging systems and/or one or more social networking systems. In one or more embodiments, communications 250 may be stored locally within the computing device (e.g., within device 110 in the case where device 110 performs the operations described herein). Architecture 200 is capable of extracting conversation elements 240 from communications 250.

Gamified health goals 245 are predetermined health objectives that are user-specific. An example of a health goal includes physical activity. Physical activity may be quantified as an amount of movement. More particularly, a health goal of physical activity may be expressed as taking “steps”. The health goal may be gamified by associating a particular number of steps with the health goal. An example of a gamified health goal may be “N steps” where “N” represents a number of steps to be taken to reach the gamified health goal. In this case, the user's goal for physical activity is taking the specified number of steps (e.g., N steps) in an enumerated time period (e.g., a day or a week).

Another example of a health goal is may be for a particular health metric such as heart rate and/or blood pressure. An example of a gamified health metric may be a particular heart rate or average heart rate and/or a particular blood pressure measurement or average blood pressure measurement. In this case, the user's goal is to perform activities that allow the user to reach the health goal such as an average heart rate of “N” beats per minute as measured over a particular period of time and/or an average blood pressure measurement as measured over a particular period of time. Other examples of health goals may relate, but are not limited to, visual acuity, mental acuity, and/or hearing acuity.

Activity map 235 specifies correlations of activities of the user with gamified health goals for the user. In one or more embodiments, the activities are user-specific activities such as activities that the user has performed in the past. In the case of a gamified health goal for physical activity such as taking a particular number of steps, examples of activities correlated with the gamified health goal include, but are not limited to, walking, getting coffee, mowing the grass, or performing other chores. Activity map 235 correlates activities that a user performs with health goals and/or gamified health goals of the user.

For purposes of illustration, consider a gamified health goal of taking 10,000 steps. In the case where the user typically mows the grass, goes for a walk, and walks to get a cup of coffee, these activities can be associated with the 10,000 step gamified health goal. Example entries in activity map 235 may include “mow-steps-2,500”, “walking-steps-2,500”, and “coffee-steps-150”. In the examples provided, each of the activities is quantified in terms of the health goal relating to physical activity and expressed as “steps”. The example entries indicate that when the user mows the grass, the user typically takes 2,500 steps. When the user goes for a walk, the user typically takes 2,500 steps. When the user gets a cup of coffee, the user typically takes 150 steps.

In one or more embodiments, the quantification of the activities may be performed by a user device over time. For example, the user may indicate an activity being performed and the device may measure the number of steps taken until the user indicates completion of the activity. The device determined number of steps may be stored in the appropriate entries of activity map 235. In one or more other embodiments, the user may simply enter a number of steps for different activities that may be stored in the appropriate entries in activity map 235.

In the case of other health goals such as heart rate and/or blood pressure, activity map may correlate activities, e.g., going to the gym, with the goal of achieving a particular health metric (e.g., heart rate and/or blood pressure). Similarly, in the case of mental, visual, or hearing acuity, activity map 235 may correlate activities, e.g., reading an article, exercise, socializing with friends, with particular gamified health goals relating to mental, visual, or hearing acuity.

In general, processor 205 is capable of matching conversation elements 240, e.g., words and/or word combinations, extracted from communications 250 with activities of activity map 235. The activities in activity map 235, in being correlated with health goals (e.g., “steps”), are also correlated with the user's gamified health goal. In this example, the system is capable of matching the term “steps” from the various entries corresponding to “mow”, “coffee”, and “walking” with the term “steps” in the gamified health goal of “10,000 steps” in gamified health goals 245, thereby establishing the correlation between the selected conversation elements 240 (and activities) and the user's gamified health goal. Processor 205 is capable of suggesting particular activities such as “mow the grass” and/or “get coffee” to the user as a way of reaching the user's gamified health goal of taking 10,000 steps.

Architecture 200 may include fewer components than shown or additional components not illustrated in FIG. 2 depending upon the particular type of device and/or system that is implemented. The particular operating system and/or application(s) included may vary according to device and/or system type as may the types of I/O devices included. Further, one or more of the illustrative components may be incorporated into, or otherwise form a portion of, another component. For example, a processor may include at least some memory.

As an illustrative example, in the case where architecture 200 is used to implement a server type of data processing system, operating system 225 may be a server-side operating system; and, application 230 may be a server-side application that, when executed, causes the server to perform the various operations described herein. In the case where architecture 200 is used to implement a device such as a client device, operating system 225 may be a client-side operating system; and, application 230 may be a client-side application that, when executed, causes the client to perform the various operations described herein. Further, devices such as mobile devices may include one or more wireless transceivers (e.g., types of network adapters) while other types of data processing systems may not.

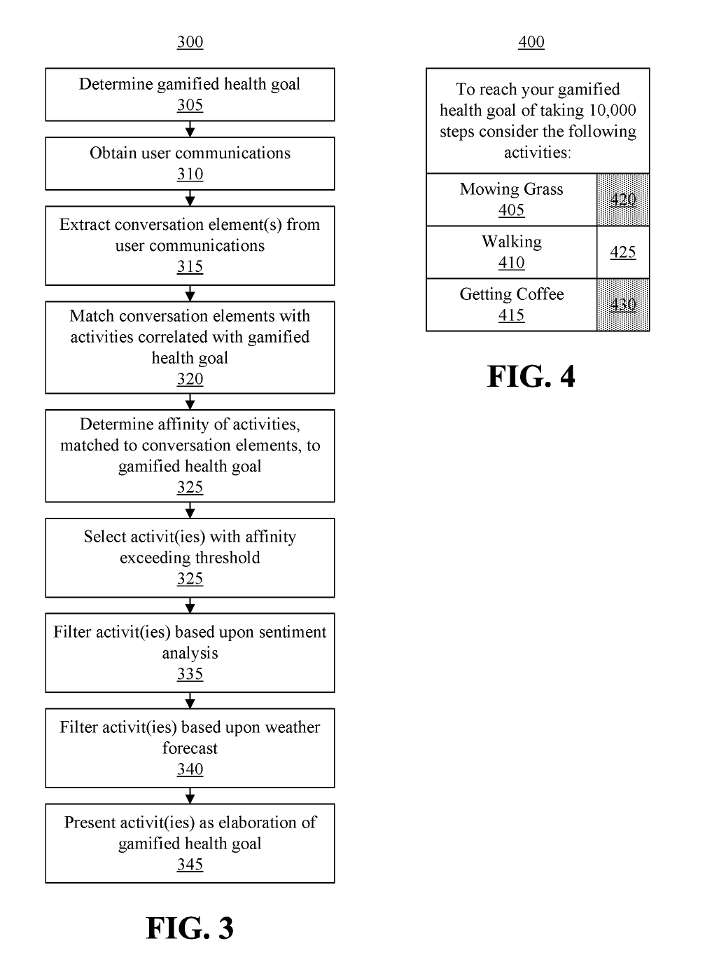

FIG. 3 depicts an example method 300 for elaborating on a gamified health goal based upon user communications. Method 300 may be performed by computer hardware (hereafter a “system”) implemented using an architecture the same as, or similar to, architecture 200 described in connection with FIG. 2. In one or more embodiments, method 300 is performed by goal achievement system 135. In one or more other embodiments, method 300 is performed by a device such as device 110 and/or device 115.

In block 305, the system determines a gamified health goal for the user. In particular embodiments, the gamified health goal is one that has been established for the user and has not yet been achieved or met by the user. The gamified health goal, for example, may be one that has been established and stored in memory. For purposes of illustration, consider the case where user A has a gamified health goal of 10,000 steps that has not yet been achieved.

In block 310, the system obtains user communications. The system is capable of retrieving communications of the user from one or more other systems. The other systems may include, but are not limited to, social networking systems, whether for professional use and/or personal use, and messaging systems (e.g., instant messaging, text messaging, SMS messaging, electronic mail). The communications, taken collectively, may be referred to as a conversation history for the user.

The system may retrieve user communications using any of a variety of techniques. In one example, the system may retrieve the entirety of a user's communications (e.g., the entire available conversation history for the user) from a given communication system. In another example, the system may retrieve the user's communications once per day, e.g., on a daily basis, from one or more communication systems. In another example, the system may retrieve the user's communications at regular time intervals such as every thirty minutes from one or more communication systems.

In one or more embodiments, the system may retrieve both private and public communications. In one or more other embodiments, the system may retrieve only public communications of the user. In particular embodiments, the system may be limited to retrieving only words from the user's communications determined to be of high importance. For example, the system may retrieve only communications having a particular designation such as those with a high importance flag in an email, starred communications, those communications processed using a particular email rule or rules, an activity stream in a social networking system having a weight (e.g., a particular weight or at least a minimum weight), or communications with other indications of importance within the subject of the message (e.g., particular words and/or symbols). In still other embodiments, the system is capable of retrieving only communications of a particular tense such as present tense, plusperfect tense, or future tense, for example.

In particular embodiments, in order for the system to access the communications of the user, the user may need to “opt in” to sharing such information. The granularity of the permissions given may vary from coarse gained to fine grained. For example, the user may be able to opt in to sharing communications from one or more different communication systems, opt in to sharing private communications on a per communication system basis, or opt in to sharing public communications on a per communication system basis.

In retrieving communications for the user, the system uses the user's identity to retrieve the conversation history of the user from one or more communication systems. The conversation history may be retrieved in text form. The conversation history may include the body portion of messages and/or any supporting content and/or metadata for such messages. In one or more other embodiments, audio, video, or multimedia content may be retrieved and translated into a textual representation using speech recognition, image processing (e.g., object and/or person recognition and/or optical character recognition as performed on images and/or video), or other suitable technologies for generating text from non-text sources.

Example 1 illustrates the body portion of a message and the supporting content and/or metadata corresponding to an electronic mail type of communication that may be received by the system in block 310.

### Example 1

{

″published″: ″2011-02-10T15:04:55Z″,
″actor″: {
″email″: ″fred@mail.com″,

″displayName″: ″Fred Lutz″

},
“summary” : “What about mowing the lawn?”
″target″ : {
″email″: ″martin@email.com″,

″displayName″: ″Martin Smith″

}

}

Example 2 shows example communications between user A engaged in a conversation with user B (e.g., a conversation history) that may be received by the system in block 310.

### Example 2

User B: Are you going to do yard work?

User A: Absolutely, it is my solitary time and the kids' play time.

User A: I plan on mowing.

In block 315, the system is capable of extracting conversation element(s) from the user's communications. The system is capable of performing NLP to identify particular conversation elements (e.g., words and/or word combinations) from the user's communications. As an illustrative and nonlimiting example, the system is capable of generation a tokenized version of the conversation history by creating N-grams.

For instance, the communication “I plan on mowing” from user A may be tokenized as shown below in Example 3.

### Example 3

NULL-I

I-plan

plan-on

on-mowing

mowing-NULL

The system is capable of performing the N-gram analysis illustrated in Example 3 for the entire conversation shown in Example 2. In one or more embodiments, the system is further capable of translating words into stem words. For example, the system may truncate or translate the term “mowing” into “mow” for purposes of analysis. In the N-gram analysis, using NLP, for example, the system is capable of inferring that the term “it” in user A's first statement refers to “yard work”. The system is capable of determining that “mowing” is the same as “yard work” or is a subset of “yard work”. The system may determine that “yard work” occurs two times (one instance of “yard work” and an instance of “it”), while the term “kids” occurs once. While Example 3 illustrates the use of bigrams, in other embodiments, the system is capable of generating unigrams, bigrams, and/or trigrams. The use of bigrams is for purposes of illustration and not limitation.

In block 320, the system determines conversation elements matching activities correlated with a health goal of the user that has not yet been achieved. In one or more embodiments, the system is capable of matching the conversation elements, e.g., the tokens or N-grams, with the activities listed in the activity map. For example, the system is capable of determining whether each of the conversation elements matches an activity from the activity map. As discussed, each activity in the activity map is correlated, or associated, with a particular health goal of the user. Referring to the prior example, the entry “mow-steps-2,500” in the activity map associates the activity of “mowing” with the health goal of “steps”. As such, the system is capable of mapping the conversation element or token “mow” to the health goal of “steps”. The system further determines that the health goal of “steps” corresponds to, e.g., matches, the gamified health goal of user A of “10,000 steps”.

Though one example match is illustrated in block 320, it should be appreciated that based upon the amount of text being processed, there may be more than one activity determined to match conversation elements for the gamified health goal identified in block 305. Thus, more than one activity may be determined from the activity map as matching selected ones of the conversation elements.

In block 325, the system is capable of determining the affinity, of activities matched to conversation elements in block 320, to the gamified health goal. In one or more embodiments, the system determines affinity of the activities based upon frequency of the matched conversation elements in the communications. The system is capable of calculating affinity so that those conversation elements corresponding to, or matching, an activity associated with the gamified health goal that occur with greater frequency result in the activity having a higher affinity.

As an illustrative and non-limiting example, the system may determine that the conversation element “mow” occurs in the communications or in a particular communication with a frequency of 5. The system may determine that the conversation element “walk” appears in the communications or the particular communication with a frequency of 1. The system determines that the affinity of the activity “mow” to the gamified health goal of “10,000 steps” is higher than the affinity of the activity “walk” to the gamified health goal. For purposes of illustration, the affinity of “mow” may be “5” while the affinity of “walk” is 1.

In one or more other embodiments, the system is capable of determining affinity as a statistical significance of the conversation elements based upon overlaps with a grammar. In general, a grammar is a data structure that specifies constraints and/or rules relating to possible sequences of symbols (e.g., conversation elements) to form meaningful sentences. In particular embodiments, an activity associated with the gamified health goal may be associated with a grammar. The system determines affinity based upon the amount of overlap of the conversation elements with the grammar associated with the activity. Based upon the amount of overlap, the system calculates affinity of the conversation elements to the gamified health goal correlated with the activity (e.g., where higher overlap corresponds to higher affinity).

In one or more embodiments, the system is capable of determining affinity (e.g., frequency) based upon an amount of overlap occurring in N-gram frequencies. For example, the system may generate unigrams, bigrams, and trigrams and determine the amount of overlap of the N-grams with activities of the gamified health goal, where the amount of overlap indicates frequency and is used to calculate affinity of the activities where greater overlap (frequency) results in greater affinity of the activity to the gamified health goal.

In one or more embodiments, the system is capable of building a more complex model that the system may use in determining affinity. The affinity may be calculated as a probability that a given activity matched to conversation elements is related to a particular gamified health goal using a statistical model and regression analysis. The frequency may be determined, for example, using a classification technique or a clustering technique. The different examples of determining affinity described within this disclosure are provided for purposes of illustration and not limitation. In other embodiments, other language processing and/or statistical processing techniques may be used. In other example, the system is capable of calculating affinity using various combinations of the techniques described and/or different weightings and/or normalizations of frequency.

In block 330, the system selects the activity (or activities) that has an affinity exceeding a predetermined or established affinity threshold. For example, the system is capable of comparing the affinity of each activity with the affinity threshold. The system selects those activities having an affinity to the gamified health goal that exceeds the affinity threshold. The system is capable of using the affinity threshold to avoid suggesting activities having a low degree of association with the gamified health goal.

Referring to the prior example, the system may determine that the conversation element for “mow” has an affinity of 0.9 with the gamified health goal of “10,000 steps”. For purposes of illustration, the affinity threshold may be set to 0.75. In this example, the system determines that the conversation element of “mow” does have an affinity exceeding the threshold affinity. Using the affinity threshold allows the system to avoid suggesting activities that while correlated with the gamified health goal, are only mentioned in passing or are tangential to the conversation. For example, the user is more likely to perform a given activity when the subject of the conversation focuses more heavily on that activity as opposed to the activity being mentioned in passing or among many other activities.

In performing block 330, the selected conversation elements are associated with a particular activity that may be performed by the user in order to reach the gamified health goal. As an illustrative and non-limiting example, the system may have identified activities such as “mow”, “coffee”, and “walking” based upon the matching of conversation elements of the user's communications with the activities in the activity map. For instance, based upon the matching conversation elements, the system may determine that one or more activities performed by the user have an affinity exceeding the affinity threshold for the gamified health goal of “10,000 steps”. Accordingly, the system effectively compiles a list of activities that may be performed by the user in furtherance of reaching the user's as yet unattained gamified health goal. The list of activities, from block 330, are activities matched to conversation elements and determined to have an affinity to the gamified health goal that exceeds the affinity threshold.

In block 335, the system optionally filters the activities based upon sentiment analysis. For example, of the activities compiled from block 330, the system is capable of determining a sentiment for each of the activities. The system is capable of removing those activities from the list that do not have a positive sentiment. For example, a positive sentiment may be a sentiment value or measure that that exceeds a sentiment threshold. By filtering the activities from the list based upon sentiment, the system avoids providing or suggesting activities to reach the user's gamified health goal that the user may disfavor. The system is able to provide or suggest those activities for reaching the gamified health goal for which the user has expressed positive sentiment and is likely to want to engage in.

In one or more embodiments, the system is capable of performing sentiment analysis on the communications of the user. The system is capable of assigning the sentiment determined for a given communication, sentence, or statement to the conversation elements extracted from such communication, sentence, or statement. The system further ascribes or assigns the sentiment of the conversation elements to the particular activities in the activity map found to match the conversation elements. In this manner, the system is capable of determining and maintaining user sentiment for the various activities listed in the activity map. In particular embodiments, the system is capable of storing the sentiment for activities in a separate list or data structure or within the activity map (e.g., within the entries of the activity map).

In block 340, the system optionally filters the activities based upon a weather forecast. The system is capable of determining whether the remaining activities to be suggested for reaching the gamified health goal comply with a weather forecast that may be obtained from a data service. As an illustrative and non-limiting example, particular activities in the activity map may be specified as being indoor or outdoor. In another example, activities specified in the activity map may be associated with particular weather attributes that are preferred and/or necessary for the activity to be performed. For example, mowing the grass (e.g., “mow”) may be associated with a “clear” weather forecast or with attributes of a weather forecast indicating that rain or other precipitation is unlikely. If mowing is on the list and the forecast indicates rain, for example, the system may remove mowing from the list of activities that may be suggested to the user to reach the gamified health goal. As another example, getting coffee may be specified as an indoor activity in the case where the user goes from an office at work to a cafeteria to get coffee. In that case, the system may keep getting coffee on the list regardless of the weather forecast.

In block 345, the system presents the activity and/or activities remaining on the list as an elaboration of the user's gamified health goal. The activities are presented as actionable events for achieving the user's as yet unachieved gamified health goal. Continuing with the prior example, the system is capable of presenting the “10,000 step” gamified health goal to the user as a list of suggested activities including “mow”, “walk”, and “coffee” that, if performed, either allow the user to achieve the gamified health goal or move the user closer to achieving the gamified health goal. The system effectively manipulates the presentation of the user's gamified health goal by breaking the gamified health goal into one or more activities presented as actionable events.

While aspects of method 300 such as affinity determination, sentiment analysis, and weather forecasts are illustrated as separate blocks, in one or more other embodiments, such operations may be performed and utilized as part of a single, larger cost function that the system may calculate and use to select and/or rank activities that may be suggested to the user to reach the gamified health goal. Those activities of the highest rank, for example, may be selected for presentation to the user. In illustration, properties such as affinity, sentiment, and relationship to weather forecast may be quantified and multiplied by a factor or weight that is specific to each respective property. The system may sum the results to generate the final score for each activity. The weighting allows the importance of the different types of analysis to be adjusted.

In accordance with the inventive arrangements described within this disclosure, in response to detecting the unachieved and gamified health goal, the system is capable of developing new exercise goals. In one or more embodiments, the system is capable of aggregating activity maps of other users (e.g., users B, C, D, E, etc.) to determine specific health goals for user A. If the system determines that the exercise goals from other users are feasible for user A, the system may present the new exercise goal(s) to user A. This allows the system to suggest activities to user A other than activities that user A has performed in the past. The system, in determining that the exercise goals are feasible, is also capable of accounting for person-specific constraints.

In one or more embodiments, the system is capable of determining feasibility of health goals from other users for a given user (e.g., user A), based upon person-to-population similarity using overlapping features. For example, health goals of other users (e.g., user B) and user A may be represented using vectors from a feature set. The system is capable of determining similarity based upon a comparison of a vector for a heath goal of user B with the vector of the health goal of user A. In response to determining a similarity between the vectors (a similarity score exceeding a threshold), the system is capable of determining that the health goal of the user B is be feasible for user A.

In one or more embodiments, the system is capable of determining feasibility based upon an established feedback loop where the system suggests health goals of other users for user A and determines that such health goals are feasible for user A based upon whether user A accepts or rejects the suggested health goals. In further embodiments, the system is capable of determining feasibility based upon whether the health goal of another user is a more specific version of, or is more precise than, the health goal of user A. In that case, the system determines that the more precise goal is feasible for user A and may suggest the health goal to user A.

FIG. 4 depicts an example of a user interface 400 for use with one or more embodiments described within this disclosure. User interface 400 may be displayed on a device of the user. In the example of FIG. 4, user interface 400 presents the gamified health goal of “10,000 steps” for the user as a plurality of actionable events, e.g., activities, to increase the likelihood the user will reach the gamified health goal. A portable or mobile device such as a mobile phone or a wearable device such as a smart watch, for example, may display user interface 400. The actionable events may be determined by the device itself or received from a remove system such as the goal achievement system of FIG. 1.

In the example of FIG. 4, a list of activities is presented to the user via user interface 400. As shown, user interface 400 lists “Mowing Grass” 405, “Walking” 410, and “Getting Coffee” 415. In one or more embodiments, the activities are listed in text form. In one or more other embodiments, an image is displayed representing each activity. In still other embodiments, both text and images may be displayed to represent each activity. Controls 420, 425, and 430 are used to for level selection and may further indicate level selection(s) by the user for the activities listed.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting. Notwithstanding, several definitions that apply throughout this document now will be presented.

The term “approximately” means nearly correct or exact, close in value or amount but not precise. For example, the term “approximately” may mean that the recited characteristic, parameter, or value is within a predetermined amount of the exact characteristic, parameter, or value.

As defined herein, the terms “at least one,” “one or more,” and “and/or,” are openended expressions that are both conjunctive and disjunctive in operation unless explicitly stated otherwise. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together.

As defined herein, the term “automatically” means without user intervention.

As defined herein, the terms “includes,” “including,” “comprises,” and/or “comprising,” specify the presence of stated features, integers, steps, operations, elements, and/or components, but do not preclude the presence or addition of one or more other features, integers, steps, operations, elements, components, and/or groups thereof.

As defined herein, the term “if” means “when” or “upon” or “in response to” or “responsive to,” depending upon the context. Thus, the phrase “if it is determined” or “if [a stated condition or event] is detected” may be construed to mean “upon determining” or “in response to determining” or “upon detecting [the stated condition or event]” or “in response to detecting [the stated condition or event]” or “responsive to detecting [the stated condition or event]” depending on the context.

As defined herein, the terms “one embodiment,” “an embodiment,” “one or more embodiments,” or similar language mean that a particular feature, structure, or characteristic described in connection with the embodiment is included in at least one embodiment described within this disclosure. Thus, appearances of the phrases “in one embodiment,” “in an embodiment,” “in one or more embodiments,” and similar language throughout this disclosure may, but do not necessarily, all refer to the same embodiment. The terms “embodiment” and “arrangement” are used interchangeably within this disclosure.

As defined herein, the term “output” means storing in physical memory elements, e.g., devices, writing to display or other peripheral output device, sending or transmitting to another system, exporting, or the like.

As defined herein, the term “processor” means at least one hardware circuit configured to carry out instructions. The instructions may be contained in program code. The hardware circuit may be an integrated circuit. Examples of a processor include, but are not limited to, a central processing unit (CPU), an array processor, a vector processor, a digital signal processor (DSP), a field-programmable gate array (FPGA), a programmable logic array (PLA), an application specific integrated circuit (ASIC), programmable logic circuitry, and a controller.

As defined herein, the term “real time” means a level of processing responsiveness that a user or system senses as sufficiently immediate for a particular process or determination to be made, or that enables the processor to keep up with some external process.

As defined herein, the term “responsive to” means responding or reacting readily to an action or event. Thus, if a second action is performed “responsive to” a first action, there is a causal relationship between an occurrence of the first action and an occurrence of the second action. The term “responsive to” indicates the causal relationship.

The term “substantially” means that the recited characteristic, parameter, or value need not be achieved exactly, but that deviations or variations, including for example, tolerances, measurement error, measurement accuracy limitations, and other factors known to those of skill in the art, may occur in amounts that do not preclude the effect the characteristic was intended to provide.

The terms first, second, etc. may be used herein to describe various elements. These elements should not be limited by these terms, as these terms are only used to distinguish one element from another unless stated otherwise or the context clearly indicates otherwise.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method, comprising:
extracting, using computer hardware, a plurality of conversation elements from communications of a user;
matching, using the computer hardware, the plurality of conversation elements to activities of the user that are correlated with a gamified health goal of the user;
determining, using the computer hardware, at least one of the activities matched to selected ones of the plurality of conversation elements having an affinity to the gamified health goal that exceeds an affinity threshold; and
providing, using the computer hardware, the at least one of the activities as an elaboration of the gamified health goal.

2. The method of claim 1, wherein the gamified health goal is for physical activity.

3. The method of claim 1, wherein the gamified health goal is for visual acuity.

4. The method of claim 1, wherein the gamified health goal is for hearing acuity.

5. The method of claim 1, further comprising:
determining a positive sentiment for the at least one of the activities based upon a sentiment analysis of the communications.

6. The method of claim 1, further comprising:
determining that the at least one of the activities complies with a weather forecast.

7. The method of claim 1, further comprising:
providing an image for each of the at least one of the activities.

8. The method of claim 1, wherein at least two of the activities matched to selected ones of the plurality of conversation elements have an affinity exceeding the affinity threshold, wherein the providing provides the at least two of the activities as the elaboration of the gamified health goal.

9. A system, comprising:
a memory configured to store program code; and
a processor coupled to the memory, wherein the processor, in response to executing the program code, is configured to initiate operations including:
extracting a plurality of conversation elements from communications of a user;
matching the plurality of conversation elements to activities of the user that are correlated with a gamified health goal of the user;
determining at least one of the activities matched to selected ones of the plurality of conversation elements having an affinity to the gamified health goal that exceeds an affinity threshold; and
providing the at least one of the activities as an elaboration of the gamified health goal.

10. The system of claim 9, wherein the gamified health goal is for physical activity.

11. The system of claim 9, wherein the gamified health goal is for visual acuity.

12. The system of claim 9, wherein the gamified health goal is for hearing acuity.

13. The system of claim 9, wherein the processor is configured to initiate operations further comprising:
determining a positive sentiment for the at least one of the activities based upon a sentiment analysis of the communications.

14. The system of claim 9, wherein the processor is configured to initiate operations further comprising:
determining that the at least one of the activities complies with a weather forecast.

15. The system of claim 9, wherein at least two of the activities matched to selected ones of the plurality of conversation elements have an affinity exceeding the affinity threshold, wherein the providing provides the at least two of the activities as the elaboration of the gamified health goal.

16. A computer program product, comprising:
a computer readable storage medium having program instructions stored thereon, the program instructions executable by computer hardware to initiate operations including:
extracting a plurality of conversation elements from communications of a user;
matching the plurality of conversation elements to activities of the user that are correlated with a gamified health goal of the user;
determining at least one of the activities matched to selected ones of the plurality of conversation elements having an affinity to the gamified health goal that exceeds an affinity threshold; and
providing the at least one of the activities as an elaboration of the gamified health goal.

17. The computer program product of claim 16, wherein the at least one of the activities is quantified in terms of a health goal.

18. The computer program product of claim 16, wherein the program instructions are executable by the computer hardware to initiate operations:
determining a positive sentiment for the activity based upon a sentiment analysis of the communication including the conversation element matched to the activity.

19. The computer program product of claim 16, wherein the program instructions are executable by the computer hardware to initiate operations:
determining that the at least one of the activities complies with a weather forecast.

20. The computer program product of claim 16, wherein at least two of the activities matched to selected ones of the plurality of conversation elements have an affinity exceeding the affinity threshold, wherein the providing provides the at least two of the activities as the elaboration of the gamified health goal.
